(19) United States
(12) Patent Application Publication
(10) Pub. No.: US 2016/0022625 A1
(43) Pub. Date: Jan. 28, 2016

(54) TASHINONES AND THEIR DERIVATIVES: NOVEL EXCELLENT DRUGS FOR ALZHEIMER DISEASE

(71) Applicants: Jie ZHENG, Akron, OH (US); Xiang YU, Rockville, MD (US); Qiuming WANG, Ypsilanti, MI (US); The University of Akron, Akron, OH (US)

(72) Inventors: Jie Zheng, Akron, OH (US); Xiang Yu, Rockville, MD (US); Qiuming Wang, Ypsilanti, MI (US)

(21) Appl. No.: 14/773,536

(22) PCT Filed: Mar. 6, 2014
(86) PCT No.: PCT/US2014/021083

§ 371 Date: Sep. 8, 2015

Related U.S. Application Data
(60) Provisional application No. 61/773,466, filed on Mar. 6, 2013.

(51) Int. Cl. A61K 31/343

(52) U.S. Cl. CPC A61K 31/343

(57) ABSTRACT

A method for disaggregating amyloid peptide aggregates the method comprising administering a tanshinone or a tanshinone derivatives to an amyloid peptide aggregate. The method may be useful for disaggregating amyloid peptide aggregates in a patient in need of such treatment, such as patients with Alzheimer's disease.

### CROSS-REFERENCE TO RELATED APPLICATIONS

This application claims priority to U.S. Provisional Patent Application No. 61/773,466 filed on Mar. 6, 2013 the contents of which are incorporated herein by reference.

### STATEMENT REGARDING FEDERALLY SPONSORED RESEARCH OR DEVELOPMENT

This invention was made with Government support under grant No. CBET-0952624 awarded by the National Science Foundation. The Government has certain rights in the invention.

### FIELD OF THE INVENTION

One or more embodiments relate to disaggregating amyloid aggregates with a tanshinone or a tanshinone derivative.

### BACKGROUND OF THE INVENTION

Alzheimer's disease (AD) is a progressive, age-related neurodegenerative disorder, which has affected over 26 million people worldwide. This number is predicted to increase to 106 million by 2050.

According to amyloid hypothesis, accumulation of β-amyloid peptide (Aβ) in brain is the primary causative factor for AD pathogenesis. The accumulation process of Aβ including (i) nucleation of monomeric Aβ into heterogeneous intermediates of lower-ordered dimers to hexamers, β-amyloid-derived diffusible ligands (ADDS), globulomers, annulars, and spherical aggregates via strong hydrophobic interaction (oligomerization), (ii) structural reorganization of these intermediates into cross-β-sheet rich protofilaments driven by specific hydrogen bonds (proto-fibrillation), and (iii) elongation of these protofilaments into mature fibrils via peptide elongation and/or thicker via lateral association of two or more proto filaments (fibrillation).

In principle, any step along the process of Aβ production, aggregation, and clearance can be considered as a potential therapeutic target to treat AD. The common inhibition strategies include interference with (1) expression of the Amyloid Precursor Protein (APP), (2) proteolytic cleavage of APP into β-amyloid peptides, (3) clearance of β-amyloid peptides from the system, and (4) aggregation of AP into soluble oligomers and insoluble amyloid fibrils. Although great efforts and promising progress have been made to block expression, cleavage, or clearance of β-amyloid peptides in these upstream processes, these strategies have not produced any effective pharmaceutical agent to date.

The degree of dementia and cognitive impairment in AD correlates much better with the concentration of soluble Aβ species instead of the number of insoluble amyloid fibrillar deposits. Accumulating evidences from in vivo studies have shown that soluble β-amyloid oligomers, but not monomeric Aβ, insoluble fibrils, or APP levels, are the most neurotoxic species lead to AD. Although the concrete toxic mechanism of soluble Aβ oligomers still remain elusive, it is generally accepted that the spontaneous aggregation of Aβ oligomers has deleterious effects on neuron cell functions with severe consequences for perturbing ionic homeostasis, triggering oxidative injury, and altering signaling pathways. The general inhibition of Aβ aggregation can't provide effective drugs to prevent AP toxicity due to the limit effect on the inhibition of soluble oligomeric Aβ generation.

Many inhibitors have been reported to prevent Aβ aggregation. However, the early stage oligomerization inhibitors, especially with reverse properties on the existing Aβ aggregates are merely reported.

Since neurotoxicity is mainly associated with the formation of soluble Aβ aggregates with β-sheet-rich structure, search for anti-oligomerization compounds and β-sheet-disrupting compounds provides alternative therapeutic approach to reduce, inhibit, and even reverse Aβ toxicity.

More importantly, since the misfolding and self-aggregation of amyloid peptides into β-sheet-rich amyloid fibrils is a general and characteristic process for all amyloid peptides, anti-aggregation and anti-β-sheet inhibitors against β-amyloid aggregation could potentially have a general inhibitory ability to prevent the amyloid formation of other amyloid peptides.

For clinical applications, β-amyloid inhibitors must resist premature enzymatic degradation, target specific tissues, cross the blood-brain barrier (BBB), facilitate nucleus uptake, while not inducing inflammation, toxicity, and other adverse immune responses. Additionally, it is well known that the cerebral vessels, especially capillary blood vessels, are the common places to clear β-amyloid by transporting β-amyloid from brain tissue to circulation system. Accumulation of AP on the inner wall of capillary blood vessels has been shown to cause vessel damage, resulting in the failure of β-amyloid clearance, which in turn promotes neuroinflammation and dementia in AD. Thus, search for β-amyloid inhibitors with a vessel protective ability, despite being often neglected, could lead to a promising therapy for AD treatment. Such inhibitors not only prevent β-amyloid oligomerization in the extracellular fluid and around the cerebral vessels, but also protect vessels from β-amyloid-induced damage.

U.S. Pat. App. No. 2009/0312413 discloses the mild Aβ aggregation inhibitory effect of tashinone compounds, but at concentrations above the amount that produce neurotoxicity in cultured cells.

U.S. Pat. App. No. 2004/0191334 discloses the use of tashinones as acetylcholinesterase inhibitors for the treatments of a wide variety of diseases, however the publication does not disclose the use of tashinones or their dirivatives for the purpose of disaggregating amyloid peptide aggregates.

### SUMMARY OF THE INVENTION

A first embodiment of this invention provides a method for disaggregating amyloid peptide aggregates comprising administering a tanshinone or a tanshinone derivative to an amyloid peptide aggregate.

A second embodiment provides a method as in the first embodiment, where the tanshinone or a tanshinone derivative, when administered, has concentration of less than 8 μm.

A third embodiment provides a method as in either the first embodiment or the second embodiment, where the ratio of the amyloid peptide to the tanshinone or a tanshinone derivative is from 1:1 to 1:5.

A fourth embodiment provides a method as in any of the first through third embodiments, where the ratio of the amyloid peptide to the tanshinone or a tanshinone derivative is from 1:1 to 1:3.

A fifth embodiment provides a method as in any of the first through fourth embodiments, where the ratio of the amyloid peptide to the tanshinone or a tanshinone derivative is about 1:2.

A sixth embodiment provides a method as in any of the first through fifth embodiments, where the tanshinone or a tanshinone derivative is selected from the group consisting of

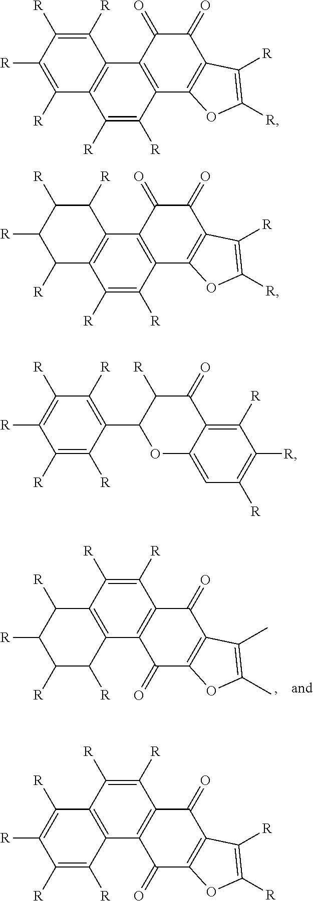

where each R is individually selected from a hydrogen atom, alcohol, amine, ester, amide, aldehyde, carboxylic acid, alkyl group, or an alkyl group with a substituted alcohol group.

A seventh embodiment provides a method as in any of the first through sixth embodiments, where a tashinone derivative is administered that includes one or more alkyl groups that increases the hydrophobic interactions with an amyloid peptide.

An eighth embodiment provides a method as in any of the first through seventh embodiments, where each R is individually selected from a hydrogen atom or an alkyl group.

A ninth embodiment provides a method as in any of the first through eighth embodiments, where each alkyl groups is a methyl group.

A tenth embodiment provides a method as in any of the first through ninth embodiments, where the tanshinone or a tanshinone derivative is selected from the group consisting of

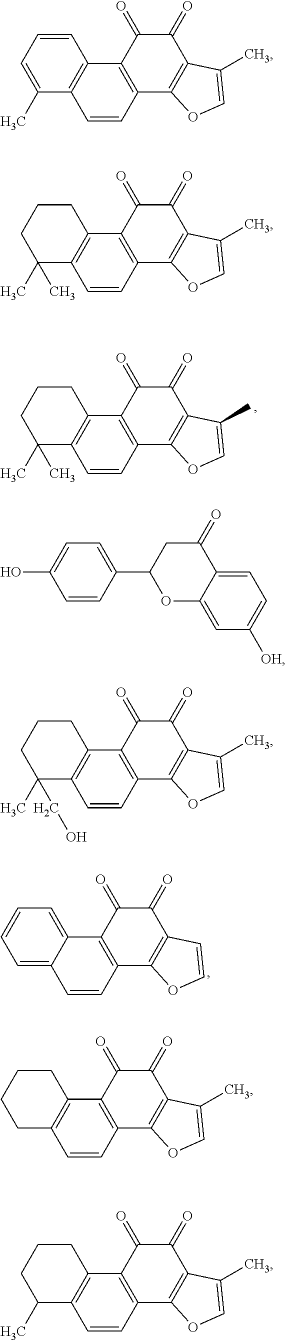

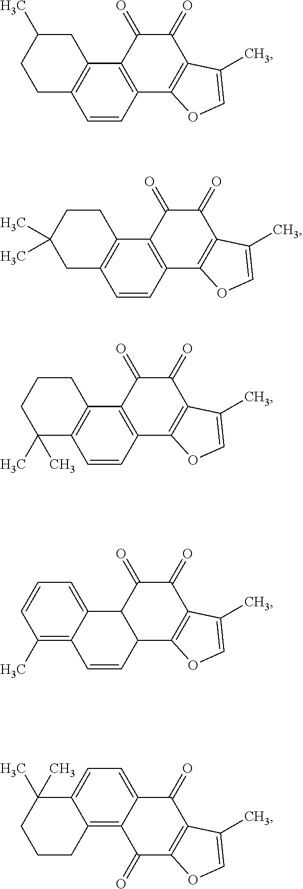 [SMILES: Cc1coc2c1C(=O)C(=O)c1c-2ccc2c1CC(C)CC2 ; Cc1coc2c1C(=O)C(=O)c1c-2ccc2c1CCC(C)(C)C2 ; Cc1coc2c1C(=O)C(=O)c1c-2ccc2c1CCCC2(C)C ; Cc1cccc2c1C=CC1c3occ(C)c3C(=O)C(=O)C21 ; Cc1coc2c1C(=O)c1ccc3c(c1C2=O)CCCC3(C)C]

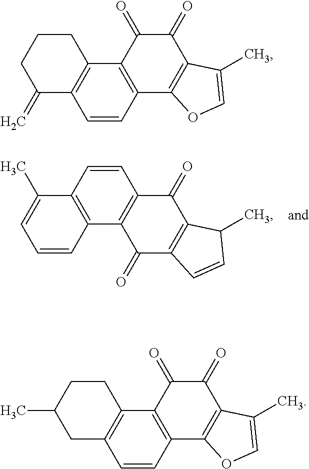

An eleventh embodiment provides a method as in any of the first through tenth embodiments, where the tanshinone or a tanshinone derivative is defined by the following formula:

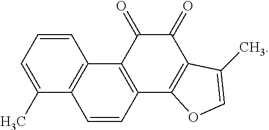 [SMILES: Cc1coc2c1C(=O)C(=O)c1c-2ccc2c(C)cccc12]

A twelfth embodiment provides a method as in any of the first through eleventh embodiments, where the tanshinone or a tanshinone derivative is defined by the following formula:

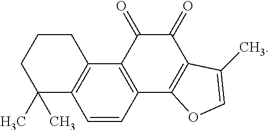 [SMILES: Cc1coc2c1C(=O)C(=O)c1c-2ccc2c1CCCC2(C)C]

A thirteenth embodiment provides a method as in any of the first through twelfth embodiments, where the amyloid aggregates include amyloids with a β-sheet structure.

A fourteenth embodiment provides a method as in any of the first through thirteenth embodiments, where the amyloid aggregates include β-amyloid peptides.

A fifteenth embodiment provides a method as in any of the first through fourteenth embodiments, the tanshinone or a tanshinone derivative is administered to an amyloid peptide aggregate within a patient.

A sixteenth embodiment provides a method for disaggregating amyloid peptide aggregates comprising administering to a patient in need of such treatment a therapeutically effective amount of a tanshinone or a tanshinone derivative.

A seventeenth embodiment provides a method as in the sixteenth embodiment where the ratio of the amyloid peptide to the tanshinone or a tanshinone derivative is from 1:1 to 1:5.

An eighteenth embodiment provides a method as in either the sixteenth embodiment or seventeenth embodiment where the ratio of the amyloid peptide to the tanshinone or a tanshinone derivative is from 1:1 to 1:3.

A nineteenth embodiment provides a method as in any of the sixteenth through eighteenth embodiments, where the ratio of the amyloid peptide to the tanshinone or a tanshinone derivative is about 1:2.

A twentieth embodiment provides the use of a tanshinone or a tanshinone derivative in the manufacture of a medicament for the treatment of an amyloid peptide aggregate.

A twenty-first embodiment provides tanshinone or a tanshinone derivative for use in treating an amyloid peptide aggregate.

### BRIEF DESCRIPTION OF THE DRAWINGS

FIG. 1A provides the chemical structure of tanshinone I.

FIG. 1B provides the chemical structure of tanshinone IIA.

FIG. 2A provides a graph of the ThT fluorescence change of Aβ aggregation in solution in compare with Aβ incubated with tanshinone I. Data represents the average of three replicate experiments.

FIG. 2B provides a graph of the ThT fluorescence change of Aβ aggregation in solution in compare with Aβ incubated with (FIG. 2B) tanshione IIA. Data represents the average of three replicate experiments.

FIG. 2C provides a graph of the ThT fluorescence change of Aβ aggregation in solution in compare with Aβ incubated with cryptotanshinone and liquiritigenin. Data represents the average of three replicate experiments.

FIG. 3A is a chart showing the time-dependent ThT fluorescence changes for Aβ1-42 incubated with tanshinones at the mole ratio of Aβ:TS=1:1, as compared to Aβ alone. Error bars represent the average of three replicate experiments.

FIG. 3B is a chart showing the time-dependent ThT fluorescence changes for Aβ1-42 incubated with tanshinones at the mole ratio of Aβ:TS=1:2, as compared to Aβ alone. Error bars represent the average of three replicate experiments.

FIG. 4 is a chart showing the disaggregation effect of TS1 and TS2 on Aβ mature fibers at a molar ratio of Aβ:TS=1:0, 1:1, 1:2, and 1:5. Error bars represent the average of three replicate experiments.

FIG. 5 is a chart showing the inhibition of Aβ-induced cell membrane disruption against SH-SY5Y cells. Cell death was determined using live/dead assay and evaluated by fluorescence change (ΔF). Data points shown are the mean±SD from three independent experiments.

FIG. 6 is a chart showing the probabilities of atomic contacts between Aβ residues and tanshinones for (a) TS1-5, (b) TS1-10, (c) TS2-5 and (d) TS2-10 systems.

FIG. 7 is a schematic model for the anti-aggregation and disassembly effects of tanshinones on Aβ amyloid formation.

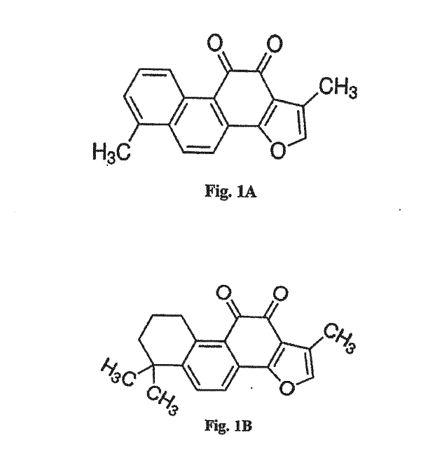

### DETAILED DESCRIPTION OF ILLUSTRATIVE EMBODIMENTS

According to one or more embodiments, a method is provided for disaggregating amyloid aggregates that comprises administering a tanshinone or a tanshinone derivative to amyloid aggregates. In these or other embodiments, a method for disaggregating amyloid peptide aggregates comprising administering to a patient in need of such treatment a therapeutically effective amount of a tanshinone or a tanshinone derivative.

In order to exhibit the therapeutic effective properties, the tanshinones or tanshinone derivatives need to be administered in a quantity sufficient to disaggregate amyloid peptide aggregates without introducing neurotoxicity to cells. This amount can vary depending upon the particular disease or condition being treated, the severity of the patient's disease or condition, the patient, the particular compound being administered, the composition or presence of any excipients, the route of administration, and the presence of other underlying disease states within the patient, etc. A proper dosage of these compounds can be readily determined using a standard dose-response protocol. The tanshinones or tanshinone derivatives typically are effective in concentration from about 4 μm to 8 μm. It has been found that compositions greater than 8 μm lead to significantly higher cell death.

Advantageously, it has been found that tanshinone or tanshinone derivatives can disaggregate amyloid peptide aggregates by binding to hydrophobic β-sheet groves present in amyloid oligomers. The amount of tanshinone or a tanshinone derivative administered may be described in reference to the amount of amyloid peptide present. In one or more embodiments, the molar ratio of tanshinone or a tanshinone derivative to amyloid peptide may be greater than 0.01:1, in other embodiments greater than 0.1:1, and in still other embodiments greater than 1:1. In one or more embodiments, the molar ratio of tanshinone or a tanshinone derivative to amyloid peptide may be less than 5:1, in other embodiments less than 4:1, and in still other embodiments less than 3:1. In one or more embodiments, the molar ratio of tanshinone or a tanshinone derivative to amyloid peptide may be from 0.01:1 to 5:1, in other embodiments from 0.1:1 to 4:1, and in still other embodiments from 1:1 to 3:1.

Amyloid peptide aggregates adopt polymorphic structures including β-sheet-rich oligomers, unstructured oligomers, or fibrils. Among the structures, β-sheet-rich oligomers are likely to be the most toxic species. Tanshinones and tanshinone derivatives exhibit strong β-sheet binding and β-sheet disrupting ability to Aβ oligomers. The strong β-sheet binding and β-sheet disrupting ability is non-specific, and allows tanshinones and tanshinone derivatives to disaggregate amyloid peptide aggregates of varying structures. Examples of amyloid peptides include Aβ and its fragments, hIAPP and its fragments, α-synuclein, and prion.

Tanshinones are lipophilic compounds extracted from the roots of Salvia miltionhiza Bunge (which may be referred to by its traditional Chinese herbal medicine name of danshen). Naturally occurring tanshinones that may be extracted from Salvia miltionhiza Bunge include tanshinone I and tanshinone IIA.

A tanshinone derivative may be formed by replacing one or more substituents onto the base structure of a tanshinone molecule. Tanshinone derivatives may also include therapeutically acceptable salts of a tashinones or substituted tanshinone compounds. In one or more embodiments, a tanshinoderiviative may be defined by formula I or a therapeutically acceptable salt thereof:

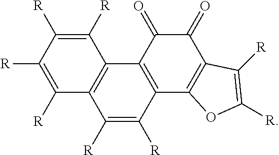

where each R is individually selected from a hydrogen atom, alcohol, amine, ester, amide, aldehyde, carboxylic acid, alkyl group, or an alkyl group with a substituted alcohol group. In one or more embodiments, a tanshinone deriviative may be defined by formula II or a therapeutically acceptable salt thereof:

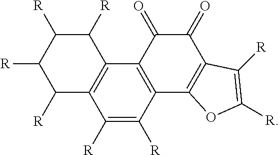

where each R is individually selected from a hydrogen atom, alcohol, amine, ester, amide, aldehyde, carboxylic acid, alkyl group, or an alkyl group with a substituted alcohol group. In one or more embodiments, a tanshinone deriviative may be defined by formula III or a therapeutically acceptable salt thereof:

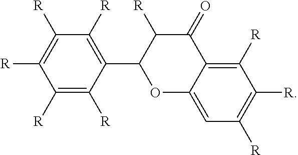

where each R is individually selected from a hydrogen atom, alcohol, amine, ester, amide, aldehyde, carboxylic acid, alkyl group, or an alkyl group with a substituted alcohol group. In one or more embodiments, a tanshinone deriviative may be defined by formula IV or a therapeutically acceptable salt thereof:

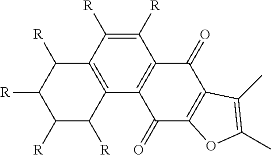

where each R is individually selected from a hydrogen atom, alcohol, amine, ester, amide, aldehyde, carboxylic acid, alkyl group, or an alkyl group with a substituted alcohol group. In one or more embodiments, a tanshinone deriviative may be defined by formula V or a therapeutically acceptable salt thereof:

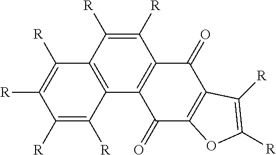

where each R is individually selected from a hydrogen atom, alcohol, amine, ester, amide, aldehyde, carboxylic acid, alkyl group, or an alkyl group with a substituted alcohol group.

In one or more embodiments, an alkyl group suitable as a substitute in formulas I, II, III, IV, or V may be a cyclic, linear, or branched alkyl groups.

In one or more embodiments, an alkyl group with a substituted alcohol group suitable as a substituent in formulas I, II, III, IV, or V may be a cyclic, linear, or branched alkyl groups with one or more hydrogen atoms replaced with an alcohol group. Specific examples of suitable alkyl groups with a substituted alcohol group include, but are not limited to, CH3OH groups.

In one or more embodiments, a tashinone derivative may be prepared with increased hydrophobicity over the naturally occurring tashinone. Increased hydrophobility will enhance hydrophobic interactions with amyloid peptides, preventing amyloid aggregation and/or causing amyloids to disaggregate. In these or other embodiment, the tashinone derivative may include one or more alkyl groups. Suitable alkyl groups for increasing the hydrophobic interactions include, but are not limited to, CH3 and (CH₂)ₙCH₃ where n=1-3, groups.

In one or more embodiments, tashinone or tashinone derivative may be defined by formulas I, II, III, IV, or V where each R is individually selected from a hydrogen atom or an alkyl group.

Specific examples of tashinones or tanshinone derivatives include, but are not limited to

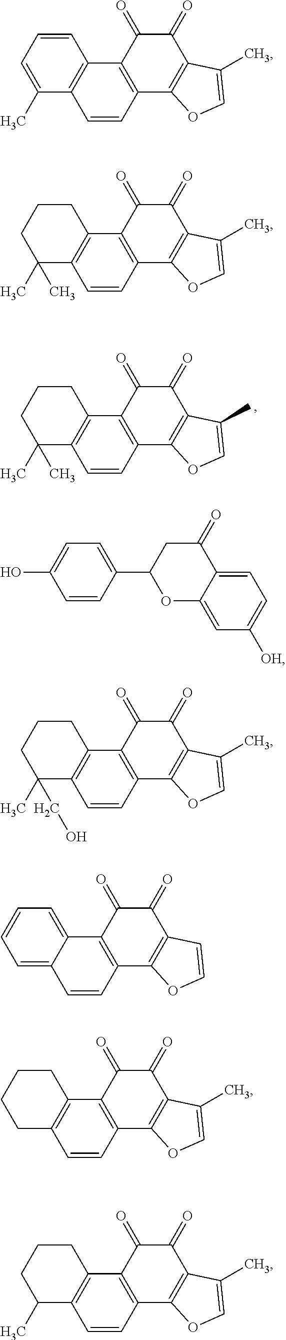

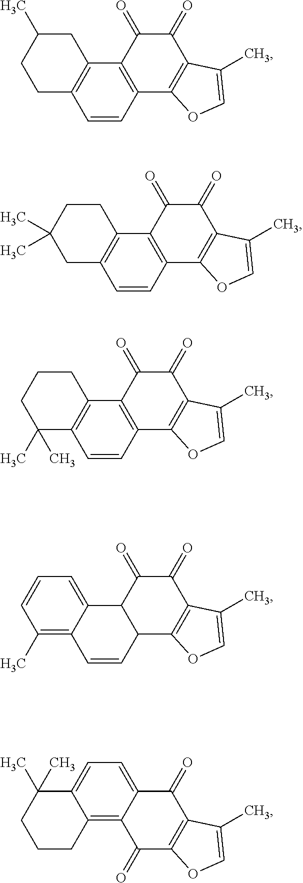 [SMILES: Cc1coc2c1C(=O)C(=O)c1c-2ccc2c1CC(C)CC2 ; Cc1coc2c1C(=O)C(=O)c1c-2ccc2c1CCC(C)(C)C2 ; Cc1coc2c1C(=O)C(=O)c1c-2ccc2c1CCCC2(C)C ; Cc1cccc2c1C=CC1c3occ(C)c3C(=O)C(=O)C21 ; Cc1coc2c1C(=O)c1ccc3c(c1C2=O)CCCC3(C)C]

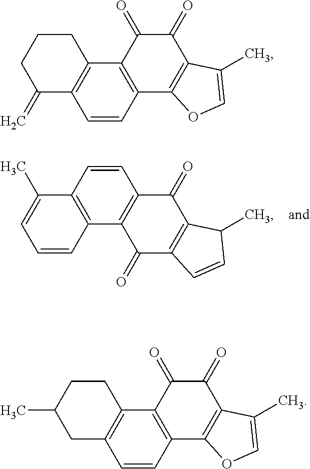

In one or more embodiments, the tanshinone or tanshinone derivative may be included in a pharmaceutical composition. Pharmaceutical compositions may include a tanshinone, a tanshinone derivative, or a pharmaceutically acceptable salt thereof and at least one pharmaceutically acceptable excipient. The pharmaceutical compositions include those suitable for subdermal, inhalation, oral, topical or parenteral use. Examples of pharmaceutical compositions include, but are not limited to, tablets, capsules, powders, granules, lozenges, or liquid preparations.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Pharmaceutical compositions may contain from 0.1% to 99% by weight tanshinone, tanshinone derivative, or pharmaceutically acceptable salt thereof depending on the method of administration.

In light of the foregoing, it should be appreciated that the present invention significantly advances the art by providing a method for disaggregating amyloid peptide aggregates that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Tanshinones Inhibit Amyloid Formation by Aβ In Vitro

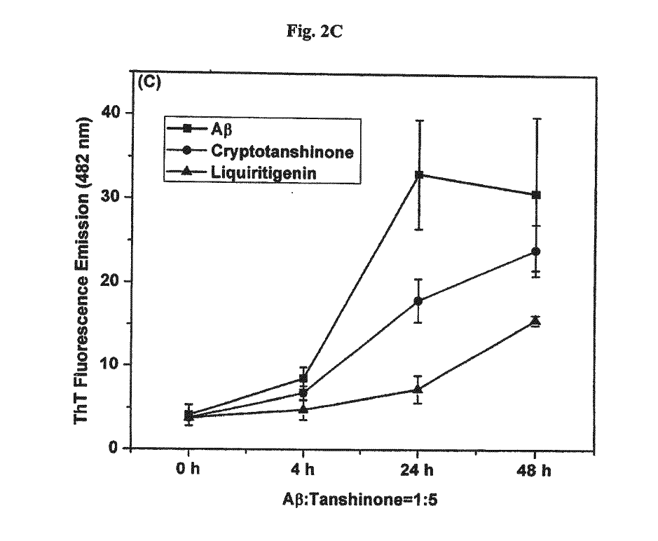

To examine the inhibitory effect of TS1 and TS2 on Aβ aggregation, the kinetics and morphological changes of Aβ1-42 amyloid formation in the presence of different molar ratios (Aβ:TS) of two tanshinone compounds were monitored by ThT fluorescence assay and AFM (Atomic force microscopy). 20 μM Aβ1-42 solution (with or without tanshinone) was incubated at 37° C. for 48 h. ThT fluorescence assay has been widely used to detect the formation of amyloid fibrils because the binding of thioflavin dyes to amyloid fibrils enables to reduce self-quenching by restricting the rotation of the benzothiozole and benzaminic rings, leading to a significant increase in fluorescence quantum yield. For Aβ aggregation only, the ThT-binding assay (FIGS. 3A,3B) and AFM images showed that within 4 hour, fluorescence signals slightly increased, accompanying with the formation of very few short and unbranched protofibrils of 7-8 nm in height. After 24 hour reaction, a strong ThT emission was observed and remained almost unchanged within statistic errors between 24 and 48 hour incubation. AFM images of pure Aβ samples without inhibitors revealed extensive long and branched fibrils with average height of 12-15 nm and average length of 1.5 μm.

In the presence of equimolar ratios of tanshinone-derived compounds (i.e. Aβ:TS=1:1), both TS1 and TS2 showed an increased lag time at the lag phase and a reduced maximum fluorescence intensity at the following growth phase. Specifically, within the first 4 hours, no fluorescence change and no protofibrils were observed by ThT and AFM, respectively. AFM images showed some small spherical particles of 1-3 nm in the Aβ-TS1 samples and of 1-6 nm in the Aβ-TS2 samples, suggesting that TS1 has stronger inhibitory potency than TS2 at the early lag phase. As reaction time increased, the ThT intensity relative to Aβ samples without inhibitors was decreased by 78.2% at 24 hour and 65.8% at 48 hour for TS1-Aβ systems, as well as by 44.8% at 24 hour and 34.6% at 48 hour for TS2-Aβ systems, respectively. To confirm the ThT results, the AFM images also revealed that TS1 generated very few and thin fiber-like materials, while TS2 produced more short thicker structures and some amorphous materials.

When the molar ratio of Aβ:TS increased to 1:2, inhibition effect of both compounds became even more pronounced. The lag phase times were prolonged. The final ThT fluorescence intensity at 24 hour was reduced by 88.5% for TS1 and by 83.4% for TS2, and no significant fluorescence change was observed between 24 and 48 hour (FIGS. 3A,3B). Consistently, the AFM images showed that with TS1 treatment, neither amorphous aggregates nor detectable amyloid fibrils were observed upon 48 hour incubation. For the Aβ-TS2 samples, unlike the formation of arrays of fibril-like materials at 1:1 molar ratio, the presence of TS2 with a higher concentration led to some small spherical species and few thin protofibrils. Taken together, ThT and AFM data clearly demonstrates that both TS1 and TS2 enable to inhibit amyloid formation by Aβ at the early lag phase and at the later growth phase, in which TS1 exhibits stronger inhibition effect on Aβ aggregation than TS2. This finding suggests that both compounds are able to bind to monomers, intermediates, and mature fibrils to interfere with structural conversion from random coils to β-structures or the association of additional monomers with the existing amyloid species to form large aggregates. Since TS1 and TS2 contain an aromatic ring structure similar to other typical organic Aβ inhibitors, it is likely that tanshinone interacts with aromatic residues of Aβ to form n-n stacking arrangement between tanshinone and Aβ. Moreover, the planar conformation of tanshinones also provides geometrical preference to align with the hydrophobic groove of amyloid fibrils, which possess an in-register organization of side chains in the regular cross-β-sheet structure. All of these effects could be attributed to the inhibition of Aβ aggregation.

### Tanshinones Disassemble Aβ Fibrils In Vitro

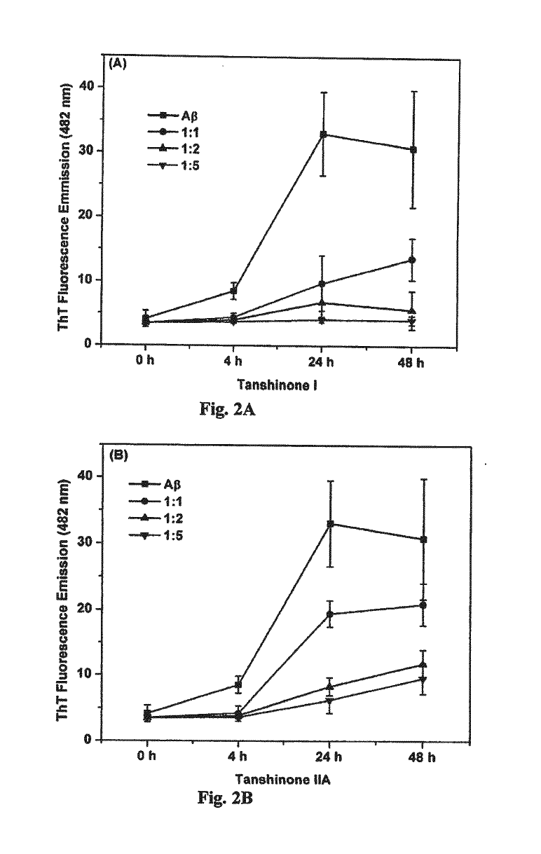

The alternative potential therapeutic strategy to treat AD (Alzheimer's Disease) is to reduce the amount of the existing amyloid plaques. A very few molecules have been reported to disaggregate Aβ amyloid. During the inhibition experiments, we observed that the ThT fluorescence signal tended to fall after the addition of tanshinone at the later stages of the fibril formation, suggesting that tanshinone may also act to reverse the aggregation process and to disassemble preformed Aβ fibrils. To determine the ability of Aβ fibril dissolution by tanshinone in vitro, we set up experiments in which tanshinone was incubated with preformed fibrils. 20 μM Aβ fibrils were first prepared by incubating Aβ monomers in solution for 48 hour, which is sufficient long enough for Aβ to grow into mature fibrils as evidenced through AFM images and ThT fluorescence (FIG. 2). Upon 48 hour incubation, Aβ fibril solution was then co-incubated with TS1 or TS2 with different molar ratios of Aβ:TS (1:1, 1:2, and 1:5) for another 48 hour at 37° C.

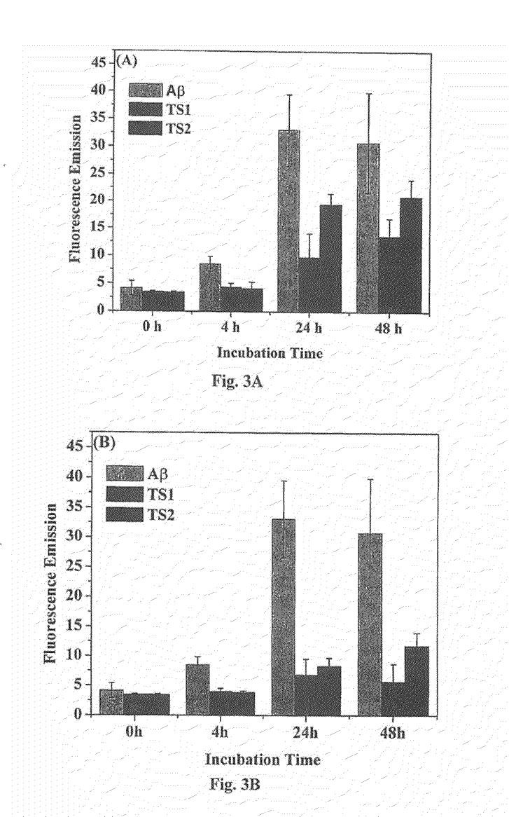

FIG. 4 shows a collection of fluorescence intensities and Aβ morphologies of the Aβ-tanshinone samples at the same time point of 48 hour, respectively. At an equimolar ratio of Aβ-TS (1:1), TS1 or TS2 only induced a subtle decrease in fluorescence (˜0.8%) as compared to the untreated control of Aβ sample (FIG. 4). Corresponding AFM images also confirmed the still existence of dense and branched fibrils with a similar morphology to control fibrils. This finding suggests that at the equimolar ratio of TS:Aβ, the dissolution process is very slow and probably only a very small fraction of Aβ dissociates from the fibrils. In contrast, when Aβ fibrils were treated with a 1:2 molar ratio of Aβ:TS, the ThT intensity was decreased by ˜30% for both TS1 and TS2, indicating the loss of the preformed amyloid fibrils that may have converted to much shorter aggregates, which did not generate an observable ThT fluorescence. Further increase of TSs concentration to Aβ:TS=1:5 showed almost no change in ThT intensity. Consistent with ThT results, AFM images also revealed similar Aβ morphologies upon the disaggregation of the preformed Aβ fibrils with 1:2 or 1:5 molar ratios. Most of the AFM chips were covered with different aggregates ranging from small spherical particles of 2-6 nm, amorphous aggregates of 3-15 nm, very few thin and short aggregates, as well as a few large and irregular-shaped deposits. The disaggregation results showed that TS1 and TS2 had comparable ability to disassemble the existing Aβ fibrils, although TS1 showed a better inhibitory potency to prevent Aβ aggregation than TS2. The sigmoidal-like dose-dependent disaggregation behavior also suggests that the dissolution of the fibrils will slow down until the formation of stable Aβ-TS complexes.

Tanshinones Protect Cultured Cells from Aβ-Induced Toxicity

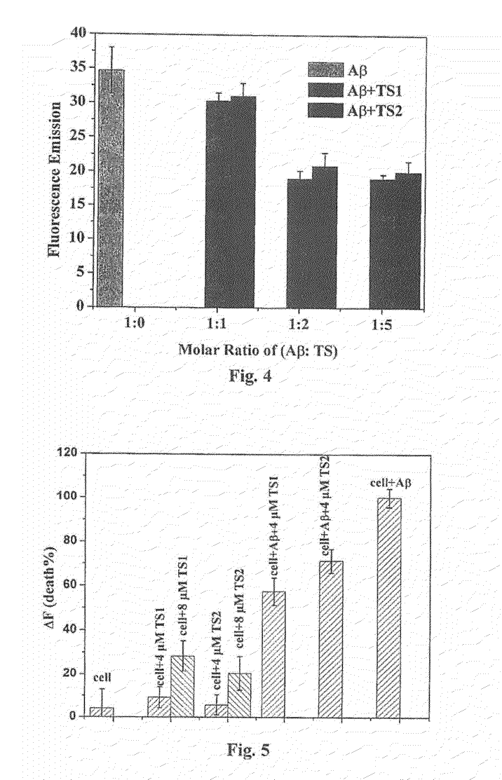

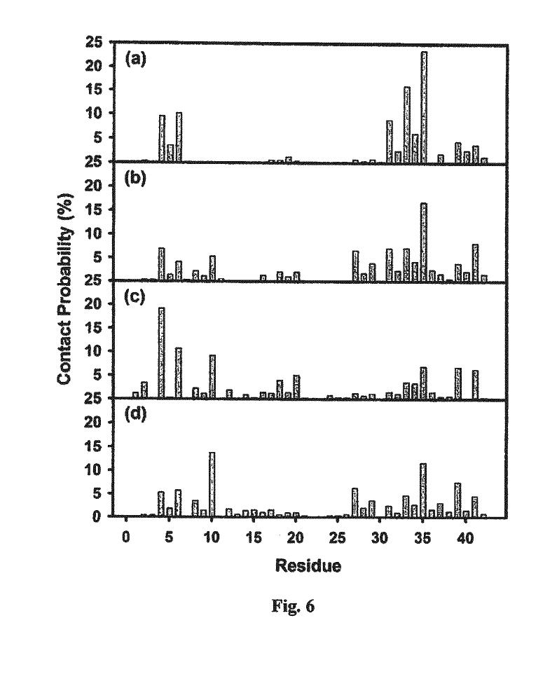

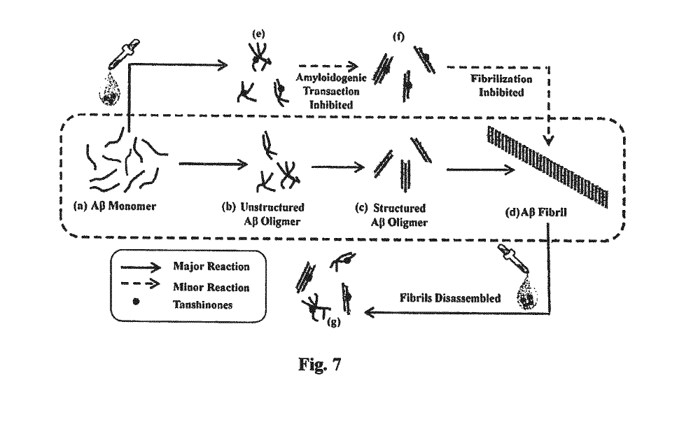

In order to determine whether tanshinone compounds are able to protect neuronal cells from toxic Aβ species, we conducted cell viability experiments to compare the toxic effects of Aβ, TS1, TS2, Aβ-TS1, and Aβ-TS2 on the SH-SY5Y cells using the SH-SY5Y live/dead assay, which is widely used in studies of Aβ toxicity. Opti-MEM reduced serum medium was used to minimize the noise of fluorescence. We then set the cell death data obtained from Aβ-cell control experiment as a basis of 100% to normalize other data for comparison. As shown in FIG. 6, incubation of SH-SY5Y cells with 20 μM Aβ for 24 hour led to significant toxicity as expected; cell death was ˜95% higher than untreated cells. When treated the cells with 8 μM TS1 or TS2 alone (Aβ:TS=1:0.4), cell death was 25.3% and 20.5% (FIG. 5), respectively. Considering the physiological concentration of Aβ is as low as 10-9 M, 8 μM tanshinone may exceed a lethal dose to induce cell death. We thus reduced the concentation of TS1 or TS2 to a clinical trial level of 4 μM for cell viability tests. As expected, the use of 4 μM TS1 or TS2 alone only induced 9.3% and 5.8% cell death, comparable to native cell apotosis of 4.3%. We then performed Aβ-tanshione cell toxicity experiments using a 1:0.2 mixture of tanshinones (4 μM) and Aβ (20 μM). It can be seen that the presence of small amount of tanshinones greatly decreased the percentage of dead cells to ˜57.5% for TS1 and ˜71.3% for TS2 (FIG. 6). TS1 appears to be less toxic than TS2 by 13.8%. Consistently, fluorescence microscopy showed that when treating SH-SY5Y cells with pure TS1 or pure TS2, no observable signs of cell death was observed, indicating the non-toxic effect of tanshinone compounds on cells at a 4 μM level. However, SH-SY5Y cells treated with a 1:0.2 molar ratios of Aβ-TS1/TS2 showed a certain degree of cell death as indicated by cell shrinkage and cell detachment from the culture substratum, but the quantity of dead cells was still much less than that of massive dead cells induced by Aβ only. Cell-toxicity experiments demonstrate a significant level of tanshione-induced cell protection, indicating that tanshione is an effective inhibitor of Aβ-induced in vitro toxicity.

Molecular Insights into Binding Mode of Tanshinones to Aβ Oligomer

The experimental results from ThT binding assays, AFM, and live/dead cell assay confirm that TS1 and TS2 compounds exhibit different inhibitory abilities to prevent Aβ aggregation and reduce cell toxicity. However, atomic details of the interactions between tanshinone compounds and Aβ peptides are not yet well elucidated. To better distinguish binding modes, sites, and affinity between TS1-Aβ and TS2-Aβ and to correlate binding information with their corresponding inhibitory ability, we performed all-atom explicit-solvent MD simulations to study the interactions of Aβ pentamer with TS1 and TS2 compounds at different molar ratios of Aβ:tanshinone from 1:1 to 1:2. For convenience, the Aβ-tanshinone systems are denoted by the type of tanshinones and the number of tanshinones. For example, TS1-5 indicates that five tanshinone-I (TS1) molecules interact with Aβ pentamer, while TS2-10 indicates that ten tanshinone-IIA (TS2) molecules interact with Aβ pentamer.

### Binding Distribution

Binding distribution population of tanshinones around Aβ pentamer, where accumulative positions of tanshinones were sampled by every 4-ps snapshots from total eight MD trajectories (Molecular dynamics trajectories). In the bound state of TS1, TS1 tended to preferentially bind to two highly populated regions of Aβ pentamer. The first binding region was located at the external side of the hydrophobic C-terminal β-sheet. Three small G33, G37, and G38 residues sitting around M35 residues formed a kink groove, which allows TS1 to strongly interact with hydrophobic C-terminal residues. Visual inspection of MD trajectories also showed that with TS1 nestling in the major groove, the relative movement of TS1 with respect to each other became much more confined. The second region was interestingly located near the N-termini of Aβ, where was enriched with two aromatic residues of F4 and H6. Due to the restricted planar geometry of aromatic rings in both tanshinones and aromatic residues of Aβ, such ordered aromatic interactions (i.e. π-π interactions) between tanshinones and Aβ peptides could mediate specific recognition and association process of Aβ peptides, resulting in the prevention of the further growth of amyloid aggregates. A number of studies have also reported that aromatic interactions play a key role in many cases of amyloid formation, but also in the self-assembly of complex supramolecular structures. As the molar ratio of Aβ:TS1 increased from 1:1 to 1:2, three additional binding regions with initially less binding probability were intensified, which were located near a U-turn region, and S8-Y10, K16-P20 residues of the N-terminal β-sheet. In the case of TS2-Aβ systems, TS2 displayed a relative broad range of binding distributions with preferential binding near the external sides of N-/C-terminal β-sheets and U-turn region similar to TS1 binding distributions. However, the binding densities of TS2 around C-terminal region were apparently lower than those of TS1. Specifically, only limited interactions between TS2 and residues of Tyr10, His14, and Phe20 were observed. Additionally, introduction of tanshinone molecules did not disturb the structural integrity of Aβ pentamer, that is, Aβ pentamer still well retained its overall and secondary structures during 40-ns simulations. As compared to experimental observation, simulation results suggest that tanshinone-induced structural disruption of Aβ oligomers should occur in a much longer timescale. Simulation results also suggest alternative Aβ inhibition pathways. Apart from that tanshinones can directly inhibit amyloid formation by breaking the performed Aβ aggregates, tanshinones also enable to bind to β-sheets to prevent lateral association of Aβ aggregates and thus to inhibit fibril growth. Simulation results confirm to the experimental observation that tanshinone can not only inhibit Aβ aggregation, but also melt the mature Aβ fibrils.

### Binding Residues

To further quantitatively identify whether TS1 and TS2 have binding preferences to certain Aβ residues, we calculated the averaged contact probabilities between each Aβ residue and tanshinones based on their atomic contacts of tanshinones within 3.5 \acute{Å} of Aβ residues (FIG. 6). Inhomogeneous contact probability between tanshinones and Aβ residues clearly indicates that tanshinones favored interactions with several Aβ residues over others. Among them, nonpolar residues, F4, H6, Y10, I31, M35, V39, and I41 generally contributed the greater binding preference to tanshinones than polar residues. Using 5% of contact probability as a threshold value, TS1 exhibited strong preferential interactions with I31 (10.1%), G33 (16.1%), M35 (23.4%), L34 (5.8%), F4 (9.7%), and H6 (10.3%). Particularly, the C-terminal residues near M35 interacted more strongly with TS1 than those N-terminal residues. For the TS2-Aβ systems, TS2 favored the interactions with I41 (6.1%), V39 (6.4%), F20 (5.6%), Y10 (9.4%), H6 (10.7%), and F4 (18.5%). Unlike TS1 showing preferential interactions with the C-terminal residues, TS2 showed strong binding preference to two hydrophobic regions of Aβ C-terminal residues near M35 and N-terminal residues of F4, H6, and Y10. Since both TS1 and TS2 contained aromatic rings. It is not surprising that that both TS1 and TS2 had preferential interactions with hydrophobic and aromatic amino acids. Particularly, F4-H6 residues near the N-termini formed an aromatic groove, while I31-M35 at the middle of C-terminal β-sheet formed a wide hydrophobic groove. Such twisted β-sheet grooves, a signature of amyloid fibrils, provide geometrical and chemical structures to specifically interact with aromatic moieties in TS1 and TS2 via n-n stacking interactions, which enable to prevent and disrupt Aβ peptide association. In fact, many of Aβ inhibitors (e.g. Congo red and thioflavin T) that share common chemical structural features such as aromatic and/or hydrogen-bonding groups were found to specifically bind to Aβ with high affinity and thus to inhibit or delay Aβ misfolding and aggregation, suggesting the importance of aromatic groups in inhibitory ability.

### Binding Sites

To gain a more quantitative comparison of binding sites between TS1-Aβ and TS2-Aβ complexes, we clustered Aβ-tanshinone complexes into different binding groups using the root-mean-square deviation (RMSD) of 5 \acute{Å} as a cutoff. FIG. 10 shows different representative binding sites derived from the top Aβ-tanshinone clusters with the highest occurrence probabilities. Structural populations for the top 5 TS1 binding sites were 30.88% at I31-M35 groove, 11.06% at F4-H6 groove, 4.28% at N27 residues, 3.77% at M35 lateral side, and 3.40% at M35-V39 groove. It can be seen clearly that the first two clusters represented the primary binding sites with a total combined binding population of 41.94% of all snapshots, while the remaining clusters presented rather diverse binding sites with relative low binding populations. In the primary binding sites, TS1 was either fitted in the C-terminal β-sheet groove formed by I31-M35 residues or aligned to aromatic residues of F4 residues. In the secondary binding sites, the populations of the remaining clusters of, and had small values of less than 5% and were similar to each other, suggesting that despite the existence of multiple binding sites, the primary binding sites are dominant. As compared to the dominant binding sites for TS1, TS2 displayed rather diverse binding sites with similar binding populations. The top 7 clustered binding sites for TS2 accounted for a total of 30.80% of all snapshots, consistent with the binding distribution of TS2. Among the top 7 binding sites, the first five binding sites were the same as TS1, with binding populations of 7.67% at I31-M35 groove, 5.06% at F4-H6 groove, 4.48% at N27 residues, 2.39% at M35 lateral side, 3.13% at M35-V39 groove, while two additional binding sites were located at Y10 residues with binding population of 5.85%, and at F20 residues with binding population of 2.22%. The existence of negatively charged residue D22 nearby F20 created a polar environment, which may help gain more atomic contacts with TS2. TS2 binding to hydrophobic I31-M35 and M35-V39 grooves had 10.80% population, which was comparable to 13.13% population as TS2 bound to aromatic residues of F4, Y10, and F20. This fact suggests that although primary dominant binding mode for TS2 is lacking, both hydrophobic and aromatic interactions still play important roles.

### Binding Affinity

TABLE 1
The binding energies (kcal/mol) for highly populated
binding sites of Aβ pentamer for TS1 and TS2.

| | Site 1 | Site 2 | Site 3 | Site 4 | Site 5 | Site 6 | Site 7 |
|---|---|---|---|---|---|---|---|
| TS1 | −18.9 ± | −18.7 ± | −17.2 ± | −10.2 ± | −16.7 ± | — | — |
| | 2.4 | 3.2 | 2.4 | 1.6 | 2.8 | | |
| TS2 | −14.7 ± | −11.7 ± | −10.1 ± | −11.0 ± | −16.1 ± | −14.4 ± | −9.1 ± |
| | 4.2 | 3.6 | 2.5 | 2.2 | 2.8 | 2.3 | 1.6 |

To quantify binding affinities between each binding site and tanshinones (TS1 and TS2), we used the GBMV method with the SASA correction to evaluate binding free energy between tanshinones and Aβ binding sites as summarized in Table 1. Comparison of binding affinities between different binding sites and between different tanshinone compounds reveals some interesting similarities and differences. First, all of binding sites provided favorable binding free energies to both TS1 and TS2. Although TS1 and TS2 shared the first five common binding sites, TS1 generally had stronger binding free energies than TS2 at the four corresponding binding site of 1, 2, 3, and 5. Second, binding affinities between different binding sites for both TS1 and TS2 were quantitatively consistent with the results obtained from binding population analysis (FIG. 6). TS1 had the most favorable binding affinities at the primary binding sites of A1 and A2, while TS2 had the relatively comparable binding affinities at most of binding sites, in which the differences in binding affinities ranged from 1.4 to 7 kcal/mol. Moreover, binding to the β-sheet groove near M35 residues (sites 1, 4, 5) by tanshinones was the relatively stronger than binding to the aromatic residues of phenylalanine and tyrosine at sites 2, 3, 6, or 7. Taken together, multiple binding sites with different binding populations and binding affinities suggests different routes to inhibit amyloid formation. Binding to β-sheet groove regions formed by I31-M35 and M35-V39 is to prevent the lateral association between different aggregates, while binding to turn or tail region is to disturb the local secondary structure of Aβ aggregates. Additionally, we also observed that TS1 and TS2 were able to stack on the top of each other to form a dimer or a trimer structure on the groove surface, which would provide additional steric energy barrier to prevent Aβ peptide association.

### Mechanistic Model for the Inhibitory Action of Tanshinones

Based on computational and experimental results, we proposed a possible model to interpret the inhibitory and disassembling effects of tanshinones on Aβ aggregation in FIG. 11. It is generally accepted that Aβ aggregation is a multiple step process, in which unstructured Aβ monomers undergo a complex conformational transition and reorganization to form intermediate oligomers and final β-sheet-rich fibrils (a→b→c→d). Both ThT and AFM results show that TSs can prolong the nucleation process, suggesting that tanshinone can bind, at least in part, to Aβ monomers to prevent peptide association (a→e) and/or to slow down conformational transition to β-structure (e→f→d). TS1 interacts stronger with Aβ monomers than TS2 during the a→e reaction, because of enhanced binding probability at the primary binding sites of hydrophobic C-terminal I31-M35 groove, which forms a steric energy barrier to prevent lateral association of Aβ peptides. Meanwhile, tanshinone could induce structural disruption to the local β-sheet of Aβ fibrils via strong binding to the turn or β-sheet groove regions of Aβ fibrils, leading to fibril disaggregation (d→g). It should be noted that due to the hydrophobic aromatic nature and planar structure of tanshione, tanshiones interact with Aβ via relatively nonspecific hydrophobic interactions with β-sheet-rich side chains. This binding mode implies that tanshione could have a general inhibition potency to prevent the aggregation of a wide range of amyloid peptides, whose aggregates adopt similar β-sheet-structures.

Small heterogeneous products resulted from Aβ aggregation are thought to be toxic species pathologically linked to the death of neurons in AD. Thus, the development of multifunctional inhibitors against Aβ aggregates is very critical for AD treatment. In this work, we report that tanshinones, main components extracted from Chinese herb Danshen, can inhibit Aβ aggregation, disaggregate Aβ fibers, and reduce Aβ-induced cell toxicity in vitro. Th-T fluorescence binding assays and AFM confirm that both TS1 and TS2 compounds inhibit unseeded amyloid fibril formation and disaggregate Aβ amyloids. TS1 has the stronger inhibition effect than TS2, but comparable disaggregate ability to TS2, which makes tanshinones as a very few small molecules that has been shown to disaggregate preformed Aβ amyloid fibrils to date. The cell viability data show that the co-incubation of Aβ with a very small amount of TSs enables to protect cultured-cells from Aβ-induced toxicity by ˜57.5% for TS1 and ˜71.3% for TS2, respectively. MD simulations (molecular dynamics simulations) further reveal atomic details of tanshinones interacting with Aβ oligomer, in which both TS1 and TS2 prefer to bind to the C-terminal β-sheet, particular hydrophobic residues I31, M35, and V39, of Aβ pentamer. Increased molar ratio of Aβ:tanshinone from 1:1 to 1:2 has little effect on tanshinone binding sites in Aβ pentamer, suggesting that a hydrophobic groove spanning across consecutive C-terminal n-strands of Aβ pentamer represents primary tanshinone binding sites to interfere with lateral association of Aβ oligomers into higher-order aggregates. Irrespective of the details, tanshinone-derived compounds presented here constitute a new class of amyloid inhibitors with multiple advantages in amyloid inhibition, fibril disruption, and cell protection, as well as their well-known anti-inflammatory activity, which may hold great promise in treating amyloid diseases. Clearly, more in vivo studies will be required to demonstrate the pharmaceutical efficacy of tanshinones in animal tests and clinical trials, as well as other amyloid diseases.

### Methods

Reagents. 1,1,1,3,3,3-hexafluoro-2-propanol (HFIP, ≧99.9%), dimethyl sulfoxide (DMSO, ≧99.9%), tanshinone I (TS1, ≧97%), tanshinone IIA (TS2, ≧98%), 10 mM PBS buffer (pH=7.4), and thioflavin T (ThT, 98%) were purchased from Sigma Aldrich (St. Louis, Mo.). Aβ1-42 peptide (≧95.5%) was all purchased from American Peptide Inc. (Sunnyvale, Calif.).

### Aβ1-42 Purification.

Aβ1-42 peptide was obtained in a lyophilized form and stored at −20° C. as arrived. In order to break the preformed Aβ aggregates into monomers, Aβ1-42 was dissolved in HFIP for 2 h, sonicated for 30 min to remove any pre-existing aggregates or seeds, and centrifuged with 14,000 rpm for 30 min at 4° C. 75% of the top Aβ solution was subpackaged and frozen with liquid nitrogen and then dried with a freeze-dryer. The dry Aβ1-42 powder was lyophilized at −80° C. and used within 2 weeks.

### Inhibition Assay

A homogeneous solution of Aβ monomers was required for inhibition tests. The purified Aβ1-42 powder was aliquoted in DMSO for 1 min and sonicated for 30 sec. The initiation of 20 μM Aβ1-42 [containing 1% (v/v) DMSO] aggregation in solution was accomplished by adding an aliquot of the concentrated DMSO-Aβ1-42 solution to 10 mM PBS buffer, followed by immediate vortexing to mix thoroughly. Aβ1-42 solution was then centrifuged with 14,000 rpm for 30 min at 4° C. to remove any existing oligomers, which 75% of the top solution was removed for further incubation or inhibition experiments. The pure Aβ1-42 solution was incubated at 37° C. as control. For Aβ inhibition experiments, 40 mM tanshinone (in DMSO) stock solution was dissolved in freshly prepared Aβ1-42 monomer solution to a final concentration of 20 μM and 40 μM (with Aβ:tanshinone molar ratio of 1:1 and 1:2), respectively. The mixed Aβ-tanshinone samples were incubated at 37° C.

### Disruption Assay of Aβ Fibrils

Aβ1-42 fibrils were prepared by incubating 20 μM Aβ1-42 monomers for 48 h, which is sufficient long enough to enable Aβ peptides to grow into mature fibrils at a saturate state. The fibril solution was then divided into aliquots for the disruption tests. To examine the effect of Aβ:tanshinone molar ratios on the extent of disruption of Aβ fibrils and to determine the minimal usage of tanshinone for more effective disruption of Aβ fibrils, 40 mM tanshinone (in DMSO) stock solution was dissolved in the Aβ fibril solution at different Aβ:tanshinone molar ratios of 1:1, 1:2, and 1:5, respectively. All the disruption samples were incubated at 37° C.

### Thioflavine T (ThT) Fluorescence Assay

Aβ1-42 fibrillization and Aβ1-42 fibril disruption in the presence and absence of tanshinones were monitored by ThT fluorescence assay. 2 mM ThT store solution was prepared by adding 0.0328 g ThT powder into 50 mL DI water. 250 μL of the 2 mM ThT solution was further diluted into 50 mL Tris-buffer (pH=7.4) to reach a final concentration of 10 μM. 60 μL of 20 μM Aβ with or without tanshinone was put into 3 mL of 10 μM ThT-Tris solution. Fluorescence spectra were obtained using a LS-55 fluorescence spectrometer (Perkin Elmer Corp., Waltham, Mass.). All measurements were carried out in aqueous solution using a 1 cm×1 cm quartz cuvette. ThT fluorescence emission intensity of each sample was recorded between 460 and 510 nm with an excitation wavelength of 450 nm. Fluorescence intensity from solution without Aβ1-42 was subtracted from solution containing Aβ1-42. Each experiment was repeated in 3 independent samples and each sample was tested quadrupled.

### Tapping-Mode AFM

The morphology change of Aβ fibrillization and disruption in the presence and absence of tanshinones was characterized by AFM. A 25 μL sample used in the Aβ1-42 ThT fluorescence assay was taken for AFM measurement at different time points to correlate Aβ morphology change with Aβ grow kinetics. Aβ1-42 solution with/without tanshinones was deposited onto a freshly cleaved mica substrate for 1 min, rinsed three times with 50 mL deionized water to remove salts and loosely bound Aβ, and dried with compressed air for 5 min before AFM imaging (atomic force microscopy imaging). Tapping mode AFM imaging was performed in air using a Nanoscope III multimode scanning probe microscope (Veeco Corp., Santa Barbara, Calif.) equipped with a 15 μm E scanner. Commercial Si cantilevers (NanoScience) with an elastic modulus of ˜40 N m⁻¹ were used. All images were acquired as 512×512 pixel images at a typical scan rate of 1.0-2.0 Hz with a vertical tip oscillation frequency of 250-350 kHz. Representative AFM images were obtained by scanning at least 6 different locations of different samples.

### Cell Culture

All chemicals for cell culture were purchased from Life Technologies unless otherwise stated. Human neuroblastoma SH-SY5Y cell (ATCC, Manassas, Va., USA) was used as model neuron and cultured in 75 cm² T-flasks (Corning) in sterile-filtered Eagles Minimum Essential Medium and Ham's F-12 medium mixed at a 1:1 ratio containing 10% fetal bovine serum (EMEM, ATCC, Manassas, Va., USA), and 1% penicillin/streptomycin in 37° C., humidified air with 5% CO₂. Cells were cultured to confluence, and harvested using 0.25 mg/ml Trypsin/EDTA solution (Lonza). Before adding Aβ and tanshinone, cells were re-suspended in Opti-MEM reduced serum medium and counted using a hemacytometer. Cells were then plated in a 24-well tissue culture plate with approximately 150,000 cells per well in 500 μl of Opti-MEM reduced serum medium, and allowed to attach for 24 hours inside the incubator.

### Live/Dead Cell Toxicity Assay

Aβ oligomers were prepared by incubating a 1 mM Aβ-PBS solution at 37° C. for 24 h. Aβ oligomers with a molar ratio of Aβ:tanshinone of 1:0.2 were added to each well to reach a final concentration of 20 μM. The cells were then left for 24 hours before cell toxicity tests. A live/dead cytotoxicity assay, which determines live and dead cells with two probes by measuring intracellular esterase activity and plasma membrane integrity, was used to obtain cell viability/toxicity data. Cells were stained by adding 2 μM of Calcein AM (Life Technologies) to distinguish the presence of live cells with a fluorescence excitation/emission of 494/517 nm, while by adding 5 μM of Ethidium homodimer-1 (Life Technologies) to distinguish the presence of dead cells with a fluorescence excitation/emission of 528/617 nm. The cells were incubated for 15 minutes with the live/dead assay contents to activate the fluorescent dyes. A Zeiss Axiovert 40 CFL inverted microscope fitted with filters at 510 nm and 600 nm was used to obtain fluorescence images of the live and dead cells. Fluorescence readings at 494/517 nm and 528/617 nm were detected using a Synergry H1 microplate reader (BioTek, Winooski, Vt.). Cell death in each incubation condition was normalized using the equation of ΔF=(Fi/FAβ)*100%, where ΔF is the percentage of cell death in different incubation conditions compared with the death of cells co-incubate with Aβ. Fi corresponds to the dead/live fluorescence signal of cells (blank control), cells co-incubate with TS1, cells co-incubate with TS2, cells co-incubate with Aβ and TS1, cells co-incubate with Aβ and TS2, and cells co-incubate with Aβ, respectively. FAβ represents the dead/live fluorescence signal of cells co-incubate with Aβ. The fluorescence signals of blank dyes were calibrated first and then subtracted from the fluorescence signals of medium solution.

### Aβ-Tanshinone Simulation Systems

Initial atomic structure of Aβ9-40 monomer was obtained from Dr. Tycko's lab by averaging NMR-derived Aβ 18-mer. Since the residues 1-8 are structurally disordered and residues 41-42 are missing, the structural coordinates of residues 1-8 and 41-42 were constructed as an extend conformation and then reassembled into the β-hairpin structure of Aβ9-40, yielding a full-length Aβ1-42 monomer with the U-shaped β-strand-turn-β-strand conformation. The Aβ1-42 monomer consisted of two β-strands, β1 (residues V1-S26) and β2 (residues I31-A42), connected by a U-bent turn spanning four residues N27-A30. D23 and K28 formed an intrastrand salt bridge to stabilize this U-bent structure. Since Aβ1-42 prefers to aggregate into pentamer and hexamer at the early assembly of Aβ42 oligomerization, Aβ1-42 pentamer was used as a typical and toxic oligomer to interact with TS1 and TS2 to determine potential binding sites and underlying inhibition mechanism. An Aβ1-42 pentamer was constructed by longitudinally stacking Aβ1-42 monomers on top of each other in a parallel and register manner, with an initial peptide-peptide separation distance of ˜4.7 \acute{Å}, corresponding to experimental data. Aβ peptide was carboxylated and amidated at the N- and C-terminus, respectively, yielding a total net negative charge of −15 e for Aβ pentamer.

Initial geometrical coordinates of TS1 and TS2 were determined and optimized at MP2/6-31G* level by Gaussian03. After geometry optimization, the partial charges of TS1 and TS2 were derived by fitting to the gas-phase electrostatic potential using the restrained electrostatic potential (RESP) method. Then, atomic structures of TS1 and TS2 obtained from Gaussian03 were subject to the ParamChem tool (https://www.paramchem.org/) to develop the force field parameters for TS1 and TS2, which are compatible with the CHARMM (Chemistry at HARvard Molecular Mechanics) general force field. The partial charges of TS1 and TS2 were further optimized by fitting the tanshinine-TIP3P water interaction energy. To validate the force field parameters of TS1 and TS2, short 2-ns MD simulations of single TS1 or TS2 in a TIP3P water box were conducted at 310 K. The bond lengths, bond angles, and torsion angles were well maintained in the MD simulations as compared to quantum mechanism structure. The topology structure, charge distribution, bonded and non-bonded parameters of TS1 and TS2 in CHARMM format were provided in Supporting Information.

Two different molar ratios of Aβ:tanshinone (1:1 and 1:2) were used to examine the concentration effect of tanshinones on Aβ-tanshinone binding interactions and underlying tanshinone inhibition mechanisms. 5 or 10 tanshinones were initially and randomly placed around Aβ pentamer at a minimum distance of 10 Å, which allows the tanshinones to unbiasedly search and sample binding sites around Aβ Each Aβ-tanshinone system was solvated in a rectangular TIP3P water box. Any water molecule within a radius of 2.4 Å from the non-hydrogen atoms of the solute (Aβ, TS1, or TS2) was deleted. Counter ions (Na⁺ and Cl⁻) were added to the simulation box to achieve electro neutrality and ionic strength of ˜100 mM.

### MD Simulation Protocol

Simulations of Aβ-tanshinone in explicit water molecules and counter ions were performed using the NAMD (National Association of Medicaid Directors) program with CHARMM27 force fields. Each Aβ-tanshinone system was first subject to 5000 steps of steepest decent minimization with position constraints on Aβ and tanshinones, followed by additional 5000 steps of conjugate gradient minimization without any position constraint. After energy minimization, the system was then gradually heated from 50 K to 500 K in 200 ps to randomize the positions of tanshinones. The systems were then equilibrated at 310 K and 1 atm for 1 ns to adjust system size and water density under NPT ensemble with the heavy-atom coordinates of Aβ and tanshinones being constrained. Then, 40-ns MD runs were conducted to examine the mutual structural dynamics and binding events between Aβ and tanshinone. Short-range VDW interactions were calculated by a switch function with a twin cutoff at 10 and 12 Å, while long-range electrostatic interactions were calculated by the particle-mesh Ewald method with a grid size of ˜1 \acute{Å} and a real-space cutoff of 14 \acute{Å}. The RATTLE algorithm was applied to constrain all covalent bonds involving hydrogen atoms, so that a time step of 2 fs was used in velocity verlet integration. Two independent simulations of each system were run to assess simulation reproducibility using different starting coordinates and velocities. MD trajectories were saved by every 2 ps for analysis.

### Aβ-Tanshinone Binding Free Energy

Binding free energies of tanshinones to Aβ pentamer were evaluated on the representative clusters using the MM-GBMV (molecular mechanism-generalized born with molecular volume) method as implemented in CHARMM. In this method, the total binding free energy in water is approximately calculated by ΔEₜₒₜ=E_{complex}−E_{Aβ}−Eₜₛ. Each energy term (E_{complex}, E_{Aβ}, or Eₜₛ) is estimated as the sum of the gas phase energy (E_{gas}) and the solvation energy (Eₛₒₗᵥ), according to Eᵢ=<ΔE_{gas}>+<ΔEₛₒₗ>, where brackets < . . . > indicate an average energy term sampled from MD trajectories. E_{gas} contains conventional bonded (i.e. bond, angle, and torsion) and nonbonded (VDW and electrostatics) interactions, as shown Eq. 1.

ΔE_{gas}=(ΔE_{bond}+ΔE_{angle}+ΔEᵢₘₚᵣₒₚₑᵣ+ΔE_{dihedral})bond+(ΔE_{vdw}+ΔE_{elec})nonbond (1)

Eₛₒₗᵥ contains polar solvation energy (Eps) and nonpolar solvation energy (Eₙₚₛ) (Eq. 2).

ΔEₛₒₗᵥ=ΔEₚₛ+ΔEₙₚₛ (2)

Eₚₛ is calculated by solving the linear Poisson-Boltzmann equation using generalized born method of the CHARMM program. Eₙₚₛ is calculated by Eₙₚₛ=γSASA, where SASA (solvent-accessible surface area) is calculated using a water probe radius of 1.4 \acute{Å} and γ is set to 0.00542 kcal/mol. The solute and solvent dielectric constants were set to 1 and 80, respectively.

1. A method for disaggregating amyloid peptide aggregates comprising: administering a tanshinone or a tanshinone derivative to an amyloid peptide aggregate.

2. The method of claim 1, where the tanshinone or a tanshinone derivative, when administered, has concentration of less than 8 μm.

3. The method of claim 1, where the ratio of the amyloid peptide to the tanshinone or a tanshinone derivative is from 1:1 to 1:5.

4. The method of claim 1, where the ratio of the amyloid peptide to the tanshinone or a tanshinone derivative is from 1:1 to 1:3.

5. The method of claim 1, where the ratio of the amyloid peptide to the tanshinone or a tanshinone derivative is about 1:2.

6. The method of claim 1, where the tanshinone or a tanshinone derivative is selected from the group consisting of 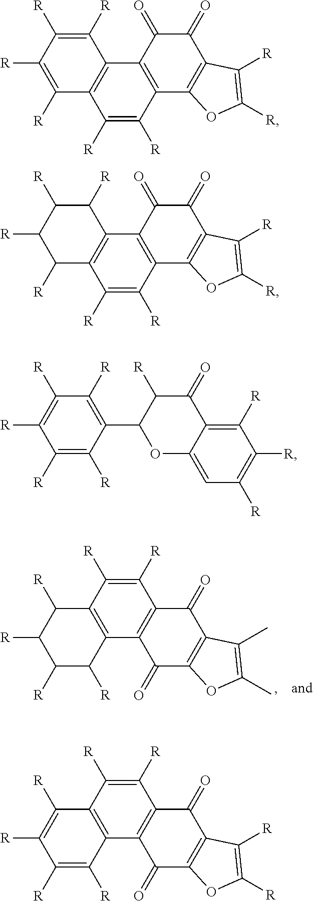
where each R is individually selected from a hydrogen atom, alcohol, amine, ester, amide, aldehyde, carboxylic acid, alkyl group, or an alkyl group with a substituted alcohol group.

7. The method of claim 6, where a tashinone derivative is administered that includes one or more alkyl groups that increases the hydrophobic interactions with an amyloid peptide.

8. The method of claim 6, where each R is individually selected from a hydrogen atom or an alkyl group.

9. The method of claim 8, where each alkyl groups is a methyl group.

10. The method of claim 1, where the tanshinone or a tanshinone derivative is selected from the group consisting of

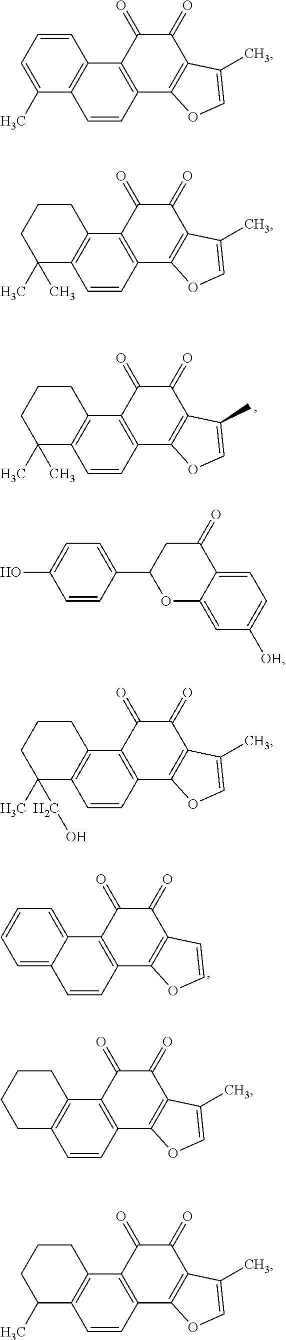

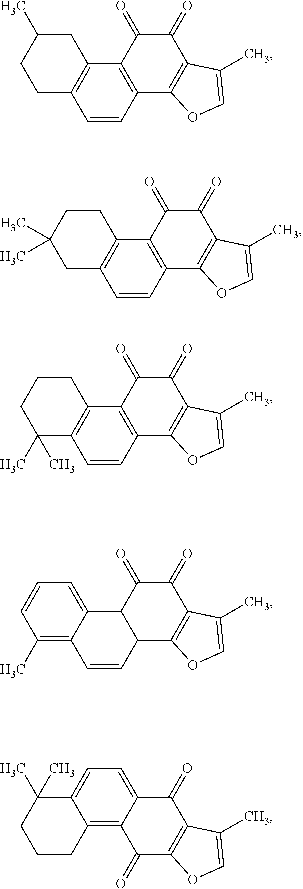 [SMILES: Cc1coc2c1C(=O)C(=O)c1c-2ccc2c1CC(C)CC2 ; Cc1coc2c1C(=O)C(=O)c1c-2ccc2c1CCC(C)(C)C2 ; Cc1coc2c1C(=O)C(=O)c1c-2ccc2c1CCCC2(C)C ; Cc1cccc2c1C=CC1c3occ(C)c3C(=O)C(=O)C21 ; Cc1coc2c1C(=O)c1ccc3c(c1C2=O)CCCC3(C)C]

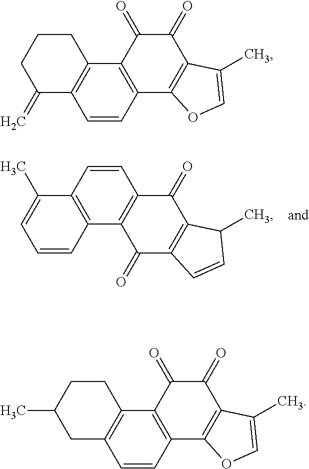

11. The method of claim 1, where the tanshinone or a tanshinone derivative is defined by the following formula:

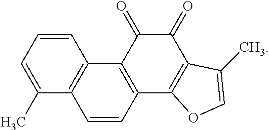 [SMILES: Cc1coc2c1C(=O)C(=O)c1c-2ccc2c(C)cccc12]

12. The method of claim 1, where the tanshinone or a tanshinone derivative is defined by the following formula:

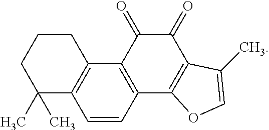 [SMILES: Cc1coc2c1C(=O)C(=O)c1c-2ccc2c1CCCC2(C)C]

13. The method of claim 1, where the amyloid aggregates include amyloids with a β-sheet structure.

14. The method of claim 13, where the amyloid aggregates include β-amyloid peptides.

15. The method of claim 1, the tanshinone or a tanshinone derivative is administered to an amyloid peptide aggregate within a patient.

16. A method for disaggregating amyloid peptide aggregates comprising: administering to a patient in need of such treatment a therapeutically effective amount of a tanshinone or a tanshinone derivative.

17. The method of claim 16, where the ratio of the amyloid peptide to the tanshinone or a tanshinone derivative is from 1:1 to 1:5.

18. The method of claim 16, where the ratio of the amyloid peptide to the tanshinone or a tanshinone derivative is from 1:1 to 1:3.

19. The method of claim 16, where the ratio of the amyloid peptide to the tanshinone or a tanshinone derivative is about 1:2.

20. (canceled)

21. (canceled)
